(19) United States
(12) Patent Application Publication
(10) Pub. No.: US 2023/0414417 A1
(43) Pub. Date: Dec. 28, 2023

(54) METHOD AND APPARATUS FOR ESTIMATING MENSTRUAL FLUID QUANTITY

(71) Applicant: Joii Ltd, Dublin (IE)

(72) Inventors: Christopher VERNALL, Dublin (IE); Andrew VERNALL, London (GB); Justyna STRZESZYNSKA, Dublin (IE)

(21) Appl. No.: 18/303,908

(22) Filed: Apr. 20, 2023

Related U.S. Application Data
(63) Continuation of application No. PCT/EP2021/079631, filed on Oct. 26, 2021.

(30) Foreign Application Priority Data
Oct. 27, 2020 (GB) 2017003.1

(51) Int. Cl. A61F 13/472; A61F 13/513

(52) U.S. Cl. CPC A61F 13/47263; A61F 13/51394; A61F 2013/8497

(57) ABSTRACT

A sanitary napkin comprising a body-facing layer comprising a body-facing surface; a liquid-impermeable garment-facing layer; and an absorbent structure therebetween; wherein the body-facing surface comprises indicia configured to facilitate the processing of data contained in an image of the body-facing surface and wherein the indicia comprise hydrophobic material. A computer implemented method for estimating menstrual fluid quantity using the sanitary napkin and a non-transitory computer-readable storage medium comprising computer-executable instructions which, when executed by at least one processor, causes the at least one processor to perform a method for estimating menstrual fluid quantity.

### CROSS-REFERENCE TO RELATED APPLICATIONS

This application is a continuation under 35 U.S.C. § 120 of International Application No. PCT/EP2021/079631, filed Oct. 26, 2021 which claims priority to United Kingdom Application No. GB 2017003.1, filed Oct. 27, 2020, under 35 U.S.C. § 119(a). Each of the above-referenced patent applications is incorporated by reference in its entirety.

### BACKGROUND

### Field of the Invention

The present invention relates to a sanitary napkin for facilitating estimation of menstrual fluid quantity, a computer implemented method for estimating menstrual fluid quantity using the sanitary napkin and a non-transitory computer-readable storage medium comprising computer-executable instructions which, when executed by at least one processor, causes the at least one processor to perform a method for estimating menstrual fluid quantity.

### Description of the Related Technology

There is a need for improved methods of estimating or measuring menstrual fluid quantity (e.g. menstrual blood loss). Existing sanitary products are often marketed to women as suitable for using when experiencing different levels of menstrual flow; often represented in terms of mL or “teaspoons” of menstrual fluid produced each day. However, this often leaves women confused about which products to use, since they cannot accurately determine how many mL or teaspoons of fluid they are producing.

This is also a concern when attempting to diagnose problematic menstrual disorders and their symptoms including heavy menstrual bleeding. A woman's own perception that she has a heavy menstrual fluid loss is the usual indication for medical and even surgical intervention. However, it is challenging for women to accurately describe the volume of blood lost. This could lead women to incorrectly assess whether or not they are suffering from menstrual disorders.

There is therefore a need for an objective method of estimating menstrual blood loss.

### SUMMARY

According to a first aspect of the present invention, there is provided a sanitary napkin comprising a body-facing layer comprising a body-facing surface; a liquid-impermeable garment-facing layer; and an absorbent structure therebetween; wherein the body-facing surface comprises indicia configured to facilitate the processing of data contained in an image of the body-facing surface and wherein the indicia comprise hydrophobic material.

The present invention, in a second aspect, provides a computer-implemented method for obtaining information related to an estimation of menstrual fluid quantity in a used sanitary napkin, the method comprising: obtaining or receiving first data representing an image of a body-facing surface of the used sanitary napkin; the body-facing surface comprising indicia configured to facilitate the processing of data contained in an image of the body-facing surface; processing the first data to generate second data identifying a location of indicia on the body-facing surface wherein the indicia comprise hydrophobic material; processing the first data to generate third data identifying a location of an area of contrasting colour on the body-facing surface; and generating an output data related to an estimation of menstrual fluid quantity based at least on the second data and the third data.

The present invention, in a third aspect, provides non-transitory computer-readable storage medium comprising computer-executable instructions which, when executed by at least one processor, cause the at least one processor to: obtain or receive first data representing an image of a body-facing surface of a used sanitary napkin; the used sanitary napkin comprising a body-facing surface; the body-facing surface comprising indicia configured to facilitate the processing of data contained in an image of the body-facing surface wherein the indicia comprise hydrophobic material; process the first data to generate second data identifying a location of indicia on the body-facing surface; process the first data to generate third data identifying a location of an area of contrasting colour on the body-facing surface; and generate an output data related to an estimation of menstrual fluid quantity based at least on the second data and the third data.

Further features and advantages of the invention will become apparent from the following description of preferred embodiments of the invention, given by way of example only, which is made with reference to the accompanying drawings.

### BRIEF DESCRIPTION OF THE DRAWINGS

FIG. 1 shows exemplary patterns, which the indicia of the invention may comprise.

FIG. 2 shows surface area saturation of a sanitary napkin relative to indicia which are in the form of visually detectable lines.

FIG. 3 shows a graph representing the relationship between volume of liquid applied to a standardized sanitary napkin relative to the normalized surface area of a contrasting colour imaged from the body-facing surface of the napkin.

FIG. 4 shows a representation of a method of using the sanitary napkin of the invention; in this example the user uses a smartphone app to image the surface of the napkin.

FIG. 5 shows a flowchart representing an exemplary process for estimating a quantity of menstrual fluid using software (e.g. a smartphone app).

FIG. 6 shows the components which may be used in an exemplary sanitary napkin of the invention.

FIG. 7 shows a graph containing the results of liquid wicking rate tests, which are described in example 3 herein.

FIG. 8 shows a graph containing further results of liquid wicking rate tests, which are described in example 3 herein.

FIG. 9 shows a graph containing the results of liquid absorptive capacity tests, which are described in example 4 herein.

FIG. 10 shows a graph containing further results of liquid absorptive capacity tests, which are described in example 4 herein.

FIG. 11 shows the hydrophobic indicia on the body-facing surface of the sanitary napkin when liquid is applied.

### DETAILED DESCRIPTION

The present invention relates to a system for obtaining information about menstruation by analysing an image of the body-facing surface of a standardized sanitary napkin after it has been used and has absorbed menstrual fluid. The sanitary napkin of the invention comprises a body-facing layer (e.g. top-sheet) comprising a body-facing surface wherein the body-facing surface comprises indicia configured to facilitate the processing of data contained in an image of the body-facing surface. In some embodiments the indicia are configured to facilitate an estimation of the quantity of menstrual fluid contained in the sanitary napkin after use. The indicia may be recognised by a computer or smartphone application and used to assist in mapping a surface area of a region of the body-facing surface which has a contrasting colour (representing menstrual fluid) to the colour of the body-facing surface; using that information to estimate a quantity of menstrual fluid contained within the sanitary napkin after use, or using the information to recommend a suitable feminine hygiene product to match the quantity of menstrual fluid being produced.

The sanitary napkin of the present invention has a body-facing layer comprising a body-facing surface; this may alternatively be referred to as a top-sheet. However, in some embodiments the body-facing layer comprises a top-sheet. The body-facing surface of the body-facing layer may also be considered as the body-facing surface of the top-sheet. When used herein, ‘body-facing surface’ of the top-sheet or body-facing layer in both cases mean the surface of the sanitary napkin exposed to the body of the user.

Information relevant to the quantity of menstrual fluid contained in a used sanitary napkin may be obtained using a standardized sanitary napkin with pre-defined structure and materials. The quantity of menstrual fluid contained in the sanitary napkin after use may be estimated by measuring the surface area of the body-facing surface which is occupied by an area of contrasting colour (e.g. stained by menstrual fluid). In the present invention an area of menstrual fluid on the body-facing surface of the sanitary napkin is identified by visually recognising and measuring an area of colour which contrasts with the colour of the body-facing surface of the body-facing layer. In other words, the surface area occupied by menstrual fluid can be recognised visually as a region having a contrasting colour to the body-facing surface. The contrasting colour of menstrual fluid typically ranges from light pink, to red and to dark brown. Sanitary napkins of the invention may be standardized by predefining the structure and materials of the sanitary napkin. A look-up table may be used to convert a measured area value for a region of contrasting colour (representing a surface area stained by menstrual fluid) to a representation of a quantity of menstrual fluid contained in the sanitary napkin. In some embodiments the quantity may be a value representing the volume of menstrual fluid (e.g. in mL) estimated to be contained in the sanitary napkin. In some embodiments the quantity may be an arbitrary designation; for example, a ‘scale of saturation’. In some embodiments the look-up table may relate to a normalised value for the area of contrasting colour with a suitable feminine hygiene product recommendation. In some embodiments, the representation of a quantity of menstrual fluid comprises at least one of (a) a volume of menstrual fluid; (b) an arbitrary designation representing quantity; and (c) a recommendation of a suitable feminine hygiene product.

The skilled person will readily appreciate how such a look-up table may be prepared for any given standardized sanitary napkin. As an example, a solution with similar consistency and flow properties to menstrual fluid is applied using a standardized procedure to a standardized sanitary napkin to be absorbed by the sanitary napkin. The solution has a colour which contrasts with the colour of the body-facing surface of the sanitary napkin such that it can be visually detected on the body-facing surface. Once absorbed into the sanitary napkin the solution stains the body-facing surface of the sanitary napkin leaving an area of contrasting colour to the colour of the body-facing surface. The surface area stained by the contrasting colour is identified and measured and this is equated with the total quantity of solution applied to the sanitary napkin. The preceding steps are repeated for a range of quantities of the solution and a look-up table for estimating quantity of solution based on the measured surface area of the contrasting colour is prepared.

The skilled person will appreciate that the consistency of menstrual fluid varies throughout menstruation, and that this will affect the flow properties of the fluid. For example, a fluid with a thinner consistency will spread further across the surface of a sanitary napkin before being absorbed into an absorbent structure/core of the napkin. The risk that menstrual fluid will leak from a sanitary napkin is greatest when the fluid has a thin consistency. Therefore, when preparing standard curves to estimate menstrual fluid quantity from the surface area of menstrual fluid, it may be preferable to use a solution which mimics the flow properties of menstrual fluid at its thinnest consistency. This will ensure, for example, that a product, which is sufficiently absorbent to prevent leakage of high spreading menstrual fluid, can be recommended to the user.

In the present invention, the body-facing surface of the body-facing layer comprises indicia configured to facilitate the processing of data contained in an image of the body-facing surface. In some embodiments the indicia may be configured to facilitate the measurement of menstrual fluid quantity contained in the sanitary napkin after use. In some embodiments the indicia are configured to facilitate estimation of the menstrual fluid quantity contained in the sanitary napkin after use, by providing objective information about the position of the sanitary napkin in an image. This allows a surface area of contrasting colour on the body-facing surface of the sanitary napkin to be measured objectively; such as relative to the indicia. For example, if an image of the body-facing surface of the used sanitary napkin is taken using a camera, the camera may be positioned a distance of 10 cm from the napkin or from the napkin. In the latter case the surface area of contrasting colour could appear smaller in the resulting image than in the former and so the estimate of menstrual fluid quantity in each case would differ. By measuring the surface area of contrasting colour relative to the indicia (e.g. normalising the surface area of menstrual fluid), the estimated menstrual fluid quantity contained in the sanitary napkin will be equivalent regardless of the distance from the body-facing surface at which the image is taken.

A computer program or software application can be designed to recognise an image of the indicia and identify the position of the sanitary napkin in an image. This allows the software to identify an area of contrasting colour (e.g. menstrual fluid) on the sanitary napkin relative to the position of indicia and convert this information to a value representing a normalised surface area of the contrasting colour on the body-facing surface. The software may then use the normalised surface area of contrasting colour to estimate a quantity of menstrual fluid and/or make a recommendation for a feminine hygiene product, using predefined data, for example, using a look-up table.

In some embodiments the indicia are visually detectable. In some such embodiments the indicia become and/or remain visually detectable when blood or menstrual fluid comprising blood is present on the sanitary napkin. In some embodiments the indicia become and/or remain visually detectable when a solution comprising a water-soluble coloured compound is applied to the sanitary napkin. The indicia may be configured to interact with menstrual fluid comprising blood, or blood, so as to become and/or remain visually detectable when menstrual fluid is present in the sanitary napkin. This allows the indicia to be visually detected (e.g. in a photographic image) on the body-facing surface of the sanitary napkin after use. In some of these embodiments the indicia do not absorb menstrual fluid and so remain visible; for example, the indicia comprise a hydrophobic material or are formed of compressed regions of material which do not absorb water-based solutions/liquids (e.g. menstrual fluid). By remaining substantially free of menstrual fluid, the indicia are visually detectable when the menstrual fluid has been absorbed by the sanitary napkin. This allows the indicia to be recognised within an image of the body-facing surface of the sanitary napkin after it has been used. By remaining visible once the sanitary napkin has been used, the indicia are capable of providing reference points for measuring the normalised surface area of menstrual fluid on the sanitary napkin.

In a further embodiment, the indicia are configured to provide a means of calibrating the position of the sanitary napkin within an image (e.g. a photographic image). In addition, or alternatively, the indicia are configured to provide a means of normalising the surface area when viewed in an image of the body-facing surface.

The indicia discussed above may be referred to elsewhere herein as ‘first indicia’. Thus, the features of the indicia discussed above apply equally when ‘first indicia’ are indicated throughout the application.

In some embodiments the sanitary napkin further comprises a wicking layer disposed between the body-facing layer and the absorbent structure. Suitably, the wicking layer is arranged to facilitate lateral movement of liquid from a region of liquid ingress (e.g. a central region of the body-facing layer) towards at least one end/distal portion of the article. In some embodiments the wicking layer is arranged to facilitate movement of liquid from a central region of the article to peripheral regions of the article, such as the end portions (e.g. the front and back portions) of the article, which are positioned at opposite ends along the longitudinal axis of the article. Consequently, including a wicking layer in sanitary napkins of the invention serves to maximise utilisation of the storage capacity of the absorbent article.

In some embodiments the sanitary napkin of the invention comprises a second set of indicia (referred to hereon in as ‘second indicia’) arranged to facilitate the processing of data contained in an image of the body-facing surface. In some embodiments the second indicia are arranged to facilitate estimation of a quantity of menstrual fluid in the sanitary napkin after use. In some embodiments the second indicia are configured to highlight a surface area of contrasting colour; for example, to enhance the visibility of the surface area of contrasting colour on the body-facing surface of the used sanitary napkin. When present, the second indicia are arranged to facilitate estimation of a quantity of menstrual fluid stored within the used sanitary napkin by enhancing the visibility of (or highlighting) the surface area of contrasting colour on the body-facing surface of the used sanitary napkin. In some embodiments the second indicia are provided on the body-facing surface of the body-facing layer and/or on the body-facing surface of the wicking layer.

In some instances, the contrasting colour provided by menstrual fluid may be less easy to detect visually, for example when the menstrual fluid contains a low concentration of blood and is therefore paler in colour. The second indicia may be arranged to concentrate the menstrual fluid at the location of the indicia to increase the visibility of an area of fluid on the body-facing surface, which improves the accuracy with which the menstrual fluid can be detected. In addition, or alternatively, the second indicia may facilitate recognition of a surface area of contrasting colour which occupies deeper layers of the sanitary napkin, such as the wicking layer. In other instance, the area of contrasting colour may be less visible on the top-sheet if the menstrual fluid has been absorbed into deeper layers of the sanitary napkin. However, the second indicia when present may highlight an area of contrasting colour after the menstrual fluid has passed through the body-facing layer. As an example, the sanitary napkin may comprise a wicking layer between the body-facing layer and the absorbent structure, wherein the wicking layer comprises a body-facing surface comprising the second indicia; when menstrual fluid enters the sanitary napkin the wicking layer conveys the menstrual fluid laterally before it is absorbed into the absorbent structure; the second indicia retain material of contrasting colour to the wicking layer and the overlying materials after the menstrual fluid has passed through the wicking layer. In an image of the body-facing surface the contrasting colour retained in the second indicia may be visually detected and so highlight the surface area through which menstrual fluid has passed.

In some embodiments the second indicia comprise a hydrophilic material. For example, the second indicia comprise a hydrophilic material which absorbs and concentrates menstrual fluid at specific loci.

In some embodiments the sanitary napkin comprises a body-facing layer comprising a body-facing surface; the body-facing surface comprising a first indicia (as described elsewhere herein); a liquid-impermeable garment-facing layer; and an absorbent structure therebetween; wherein the sanitary napkin further comprises a second indicia (as described elsewhere herein). In some such embodiments the sanitary napkin comprises a wicking layer; wherein the wicking layer comprises a body-facing surface comprising the second indicia.

Materials of Sanitary Napkin

In some embodiments the indicia comprise hydrophobic material which may be provided as a pattern of lines or spots. The lines or spots may be printed or painted onto the top-sheet using any methods known in the art.

In some embodiments the hydrophobic material comprises a hydrophobic ink. In some embodiments the hydrophobic material comprises styrene acrylate binder and ink (e.g. white ink). The hydrophobic material may be applied through printing with a paste including a hydrophobic finish or binder. The hydrophobic material may comprise hydrophobic and water-repellent agents by commercial brands including non-fluorinated finishes (e.g. by suppliers including Huntsman and Rudolf) and non-fluorinated compounds (e.g. by suppliers including Heiq, Neoseed, Organo Click, Nasiol, Nicca and Nikwax); silicon hydrophobic compounds including silicone emulsion (e.g. from Dow), silicone urethane additive (e.g. from Siltech) and polydimethylsiloxane-based compound (e.g. from Silkor); paraffin wax-based treatments (e.g from Pulcra chemicals and Schoeller Ecorepel); other wax-based treatments may be based on natural or synthetic waxes including beeswax, shellac, carnauba, candelilla, jojoba, lanolin, and montan. Hydrophobic binders may include acrylics, styrene acrylate, polyvinyl alcohol, polyvinyl chloride, polyurethane and latex; fluoropolymer treatments; surface treatments such as plasma treatment or corona discharge may be applied onto the fibres or fabrics to create hydrophobic regions.

In some embodiments the indicia comprise compressed regions of material which do not absorb water-based solutions/liquids. The compressed regions can be formed, for example, using a hot press, solder iron or hot press and aperture mesh. The optimum technique for preparing such compressed regions will depend on the material in which the compressed regions are to be formed.

### Top-Sheet

In some embodiments the body-facing layer comprises a top-sheet. In some such embodiments the top-sheet comprises a non-woven fabric including spunlaid, drylaid or wetlaid structure or a combination of these; nonwoven structures of interest include carded thermally bonded or spunbonded web; spunbond-meltblown-spunbond (SMS) structure or varieties of spunmelt composites (e.g. SMS, SMMS, SMSMMS, etc.). The nonwoven fabric may be bonded by means of thermal bonding such as calendaring, through air bonding, ultrasonic welding, chemical bonding, mechanical bonding such as hydroentanglement, or a combination of bonding mechanisms. The top-sheet may also or alternatively comprise perforated plastic films; such as low-density polyethylene, linear low-density polyethylene, ultra-low-density polyethylene, metallocene polyethylene, high-density polyethylene, polypropylene or other polyolefins, polycarbonate, polyvinyl chloride, polyester, polyvinylidene chloride, polyamides, polyurethanes, cellulose acetate, ethylene vinyl acetate, ethylene-vinyl alcohol, natural or synthetic rubbers, or regenerated cellulose cellophane. Alternatively, sustainable bio-based or biodegradable alternatives to these films may be used. In some embodiments the top-sheet comprises an embossed film; porous foams; lightweight (<50 g/m²) woven or knitted fabric; or other net/mesh structures that may be woven, knitted or constructed in an alternative method. Fibres that may be used within the top-sheet layer can include: natural fibres such as cotton, organic cotton, perforated organic cotton, bast and leaf fibres, protein fibres, bamboo or wood pulp; regenerated fibres such as viscose or lyocell; synthetic fibres including polypropylene, polyethylene, polyester, polyamides, polyacrylates, other polyolefins, bi-component fibres comprising pre-mentioned polymers; blends of natural, regenerated and synthetic fibres; synthetic fibres may contain a hydrophilic surface treatment including surfactants, plasma treatments, additives and coatings or finishes. In some embodiments the topsheet may comprise sustainable (e.g. bio-based or biodegradable) materials, for example the top-sheet may comprise polylactic acid (PLA), polyhydroxyalkanoates (PHA), 3-hydroxybutyrate-co-3-hydroxyvalerate (PHB V), polybutylene succinate (PBS), polyethylene furanoate (PEF), polytrimethylene terephthalate (PTT), thermoplastic starch (TPS), biodegradable Polyethylene terephthalate (PET), biodegradeable polypropylene (PP), polycaprolactone (PCL), polyethylene glycol (PEO), polyglycolic acid (PGA), polyvinyl alcohol (PVA), viscose, hydrophobic viscose, tencell/lyocel, cotton, hydrophobic cotton.

### Back-Sheet

The sanitary napkin of the invention comprises a liquid impermeable garment-facing layer. The garment-facing layer may comprise a back-sheet, or is a back-sheet. In some such embodiments the back-sheet comprises a plastic film; for example, low crystallinity polyethylene, polypropylene, ethylene vinyl acetate copolymers, polyurethane, polyisoprene, butadiene-styrene copolymers, styrene block co-polymers, polycarbonate, polyvinyl chloride, polyester, polyvinylidene chloride, polyamides, cellulose acetate, ethylene-vinyl alcohol, natural or synthetic rubbers or regenerated cellulose or sustainable recycled, biobased or biodegradable films. In some embodiments the back-sheet comprises a liquid impermeable nonwoven fabric. In some embodiments the back-sheet comprises a composite or laminate structure which may contain a nonwoven and/or film structures. In some embodiments the backsheet comprises sustainable recycled, biobased or biodegradable materials such as polylactic acid (PLA), polyhydroxyalkanoates (PHA), polyhydroxybutyrate (PHB), 3-hydroxybutyrate-co-3-hydroxyvalerate (PHBV), polybutylene succinate (PBS), polyethylene furanoate (PEF), polytrimethylene terephthalate (PTT), cellulose acetate, thermoplastic starch (TPS), biodegradeable polyehtylene terephthalate (PET), polyvinyl alcohol (PVA), biodegradeable polypropylene (PP), biodegradeable polyethylene (PE), polycaprolactone (PCL).

### Acquisition-Distribution Layer (ADL)

In some embodiments the absorbent article of the invention comprises an ADL. The ADL, in some embodiments comprises nonwoven fabric produced by drylaid, wetlaid or spunmelt methods. These non-woven fabrics may be bonded through thermal, chemical, or mechanical processes. Nonwoven fabrics which may be used include: carded fabric bonded by through-air bonding, hydroentanglement or needle-punching; airlaid fabrics bonded by spray chemical bonding or thermal bonding (ultrasonic or through-air); meltblown or spunbond fabrics, or a composite of both that may be bonded through calendaring. The non-woven fabric may further include embossed, creped or textured elements. In some embodiment the ADL comprises woven fabrics including 3D woven fabrics; knitted fabrics including knitted double jersey or spacer fabrics. Fibres that may be used in the above-mentioned fabrics include: natural fibres such as bast or leaf fibres and wood pulp which have been treated or untreated, cotton, treated wool; regenerated fibres such as viscose, bamboo viscose or lyocell; synthetic fibres comprising polypropylene, polyethylene, polyester (PET), polyamides, polyacrylate, other polyolefins, bi-component polymers of the pre-mentioned fibres and blends or composites of natural, regenerated, or synthetic fibres. In some embodiments the ADL comprises sustainable (e.g. biodegradeable) materials such as cotton and/or polylactic acid (PLA).

### Absorbent Structure

The absorbent article of the invention comprises an absorbent structure which acts to absorb and store liquid. In some embodiments the absorbent structure comprises an absorbent fibrous material. The fibrous material may comprise fibres with a modified cross-section to increase absorbency which may include but are not limited to splitable fibres, multi-lobal fibres, trilobal fibres, 4DG fibres (i.e. fibres comprising thermoplastic polymer, e.g. PET, PP or Nylon, which contain 6-10 grooves or channels along the longitudinal axis of the fibre) or ribbon shaped fibres; microfibres; absorbent nonwoven fabrics produced through drylaid, wetlaid or spunlaid methods with a variety of bonding mechanisms; meltblown fabrics; tissue wraps; airlaid structures; airlaid structures bonded by means of ultrasonic bonding or other thermal or chemical bonding methods; carded and needle-punched fabrics; absorbent polymeric open-cell foam made of polymers including polyurethane; absorbent sponges; woven or knitted fabrics. Fibres that may be incorporated into the structures include cellulosic wood pulp which is either treated or untreated, cotton fibres, cotton lint or cotton flock, viscose fibres with alternative cross-sections, chemically modified cellulose fibres, plant based fibres with absorbency such as hemp, flax, sodium alginate, calcium alginate, chitin, chitosan. In some embodiments the absorbent structure comprises superabsorbent polymers (SAP) in fibre or particle form; these can include sodium polyacrylate, ionic or non-ionic SAP, potassium-based SAP, citric acid-based SAP, cellulose based SAP; sulfamic acid modified starch (Cargill) based SAP; crosslinked cellulose and protein hydrogels (Tethis); absorbent gelling material; absorbents based on starch or other sustainable alternatives. In some embodiments the absorbent structure comprises sustainable (e.g. biodegradable) SAP materials such as calcium alginate, potato starch, potassium polyacrylate, carboxymethyl cellulose (CMC), chitosan and silica gel.

### Wicking Layer

In some embodiments, sanitary napkins of the invention comprise a wicking layer. Suitably, the wicking layer is arranged to facilitate lateral movement of liquid from a region of liquid ingress (e.g. a central region of a body-facing layer) towards at least one end/distal portion of the article. In some embodiments the wicking layer is arranged to facilitate movement of liquid from a central region of the article to peripheral regions of the article, such as the end portions (e.g. the front and back portions) of the article, which are positioned at opposite ends along the longitudinal axis of the article. The absorbent article may comprise a wicking layer in addition to an ADL. In these embodiments the wicking layer may be disposed between the ADL and an absorbent structure. The ADL facilitates rapid transfer of liquid away from the skin and the wicking layer facilitates lateral movement of the liquid towards peripheral portions (e.g. the end portions) of the article prior to its transfer to the absorbent structure for storage. Consequently, including a wicking layer in sanitary napkins of the invention serves to maximise utilisation of the storage capacity of the absorbent article. Inclusion of the wicking layer means that the absorbent structure may be used more efficiently to store menstrual fluid. This could allow the thickness of the absorbent structure to be reduced whilst its storage capacity remains high, resulting in a thinner sanitary napkin which remains absorbent, whilst being more comfortable for the wearer.

In some embodiments, the material used in the wicking layer has a high liquid wicking rate, which facilitates movement toward outer edges (e.g. the end portions) of the absorbent article. The wicking rate of a material may be defined in terms of its “Vertical Wicking Rate” which refers to the ability of a material to draw fluids in a vertical direction against gravity. Vertical wicking is a desirable property since it indicates a material's ability to move fluid away from a point of contact. One measure of a given material's vertical wicking performance is the Vertical Wicking Height—i.e., the distance that the material can wick liquid in a direction opposite to gravitational force. In some embodiments of the invention, the wicking layer comprises a material which exhibits a Vertical Wicking Height of at least 100 mm within 3 minutes when measured using EDANA NWSP 101.1 R0 (20) In some embodiments, the wicking layer absorbs and wicks a sufficient amount of liquid per gram of the material so as to have a liquid flux adequate to substantially move the liquid out of the insult area prior to the next liquid insult.

Test for Liquid Wicking Rate (Capillarity)

Fabrics having suitable liquid wicking rates may be identified in experiments performed in accordance with Edana Nonwovens Standard Procedure 010.1.R0(20). According to the standard test, ‘liquid wicking rate’ is a measure of the capillarity of the test material which is the rate at which a liquid is transported into the fabric by capillary action. The capillarity method measures the rate of vertical capillary rise in a specimen strip suspended in a test liquid. In some embodiments, the wicking layer comprises a material having a liquid wicking rate of at least 80 mm, optionally at least 100 m, within 3 minutes when measured using EDANA NWSP 101.1 R0 (20). The procedure for determining liquid wicking rate in accordance with this standard is as follows:
1. Cut at least 5 test specimens (30±1) mm wide×(250±1) mm long in both the machine direction and the cross direction.
2. Punch two holes, (5±1) mm diameter out of one of the short ends of each test specimen at (5±1) mm from short and long sides.
3. The liquid shall be left long enough to equilibrate with the conditioned atmosphere.
4. Clamp the test specimen vertically to the horizontal support with the punched holes at the bottom.
5. Place a glass rod through two slots to keep tension on the test specimen and maintain it vertically.
6. Place test specimen neat and parallel to the measuring rod and projecting (15±2) mm below the zero point of the measuring rod.
7. Lower the horizontal support until the zero point of the measuring rod touches the liquid surface (the lower test specimen edge is then (15±2) mm below the surface.
8. At this moment, start stopwatch.
9. Record the height of capillary rise of the liquid after 10 s, 30 s, 60 s (and 300 s if required). (If capillary rise is not a uniform straight line, record the highest point)
10. Repeat 4 to 9 with the other 9 test specimens.
11. Use fresh conditioned test liquid for each set of 10 test specimens.
12. Calculate the average capillarity rise obtained on the 5 test specimens for each specified time and the standard deviation:
   a) in the machine direction
   b) in the cross direction
13. Trace a curve using the data obtained (12), so that the wicking rate can be calculated at a required time or at a required capillary rise.

Liquid Absorptive Capacity

In some embodiments the wicking layer comprises a material which has high liquid absorptive capacity when measured using EDANA NWSP 101.1 R0 (20) According to the standard test, ‘liquid absorptive capacity’ is the mass of liquid that is absorbed by unit mass of the test material expressed as a percentage of the mass of the test material under specified conditions and after a specified time. The absorptive capacity method performed according to the above standard provides a measure of the amount of liquid held within a test specimen after specified times of immersion and drainage. This method measures the liquid stored within the test specimen itself after drainage has occurred vertically. Liquid absorptive capacity may be calculated by measuring the mass of a material after it has been submerged in 0.9% saline solution for 60 seconds and expressing the result as a percentage of the original weight of the material prior to wetting. In some embodiments, the wicking layer comprises a material having a liquid absorptive capacity of at least 600% when measured using EDANA NWSP 101.1 R0 (20). The procedure for determining liquid absorptive capacity in accordance with this standard is as follows:
1. Cut 5 test specimen (100±1) mm×(100±1) mm from the sheet. If the individual test specimen weighs less than 1 g, lay test specimens on top of each other to give to each of the piles a total stacked mass of at least 1 g.
2. The liquid shall be left long enough to equilibrate with the conditioned atmosphere.
3. Weigh the test specimen (or pile) to an accuracy of 0.01 g, using the balance and the weighing glass with cover.
4. Place the test specimen (or pile) on the stainless-steel gauze, fastening it (them) at the edges with the clips.
5. Place the gauze with the attached test specimen(s) approximately 20 mm below the liquid surface in the dish and start the stopwatch. Introduce the gauze obliquely in order to avoid trapping air bubbles.
6. After (60±1) s remove the gauze test specimen support and test specimen (or pile).
7. Remove all clips but one at one corner.
8. Hang freely vertical to drain for (120±3) s.
9. Take the test specimen (or pile) off the gauze without squeezing the liquid from it; place the test specimen in the weighing glass with cover and weight.
10. Repeat 3 to 9 for the other 4 test specimens.
11. Use fresh conditioned test liquid for each set of 5 test specimens (or piles).
12. Calculate:
   a) The liquid absorptive capacity (LAC) in % of each specimen or each pile from the following:

Mn—Mk

LAC % - - - ×100%,
Wherein Mk is mass in grams of the dry test specimen(s)
Mn is mass in grams of the wet test specimen(s) at the end of the test.
b) The average liquid absorptive capacity of the 5 test specimens (or piles) and the standard deviation.

In some embodiments the wicking layer comprises parallel laid fibres. It has been found by the inventors that materials comprising fibres which are parallel laid (meaning the fibres are mostly aligned in the machine direction) are particularly suitable for use in the wicking layer. Whilst not wishing to be bound by theory, it is believed that when the direction of the fibres is aligned with the longitudinal axis of the article, wicking towards the end portions of the article, i.e. those positioned at either end along the longitudinal axis of the article, is facilitated by capillary forces.

The inventors have also found that when the wicking layer comprises a material comprising apertures, wicking can be improved. Without wishing to be bound by theory, it is believed that introducing apertures into the material compacts adjacent fibres together, decreasing space between the fibres, which increases capillary forces and so improves wicking of liquid along bundles of the fibres. In some embodiments the wicking layer comprises a sheet of fibrous material comprising 10-40 apertures per in² (or 1-7 apertures per cm 2); for example, 15-30, or 20-25 apertures per in² (or 2-5, or 3-4 apertures per cm 2). In an exemplary embodiment, the wicking layer comprises a sheet of fibrous material comprising 22 apertures per in² (or about 3 apertures per cm 2). Suitable materials for use in the wicking layer may contain apertures of various sizes. The fibrous material comprising the apertures may alternatively be referred to as a mesh; for example, ‘22 mesh’ means there are 22 apertures per in².

Nonwoven fabrics suitable for use in the wicking layer may include drylaid, wetlaid or spunlaid formations or a composite structure of the aforementioned methods. Some potential structures may include: carded and parallel laid hydroentangled (spunlace) fabrics (with or without apertures and with or without channels or cavities); wetlaid fabric with orientation in a machine direction, hydroentangled, thermally or chemically bonded; carded and parallel laid and thermally bonded structures bonded by calendaring or through-air methods; carded parallel laid and chemically bonded structures.

In some embodiments, the wicking layer comprises a sheet of material comprising woven or non-woven fibres. In some embodiments the fibre orientation of the non-woven fibres is in a single direction or in one or more directions. Examples of suitable non-woven fibres include “spunlaces”. Spunlace is a non-woven fabric, formed by using waterjets to bond a carded web of fibres. To form a spunlace, water is emitted under high pressure and velocity from closely positioned nozzles onto a web of loose fibres. The intensity of the water stream and the pattern of the supporting drum or belt entangle, spin and curl the fibres about one another. The entangling of the fibres and the friction between these yields a cohesive web. The process makes a nonwoven fabric with physical properties of softness, high bulk, drapability, stretchiness, good strength and depending upon the fibre used, aesthetics that mimic traditional knitted or woven textiles. When the wicking layer comprises a non-woven mat of fibres, wicking along the material is improved. Without wishing to be bound by theory, it is thought that when a non-woven fibre is used, capillary forces along the fibres are not regularly interrupted as they may be in fabrics or materials which are woven in a regular pattern.

In some embodiments the wicking layer comprises biodegradable or compostable fibres. In some embodiments, the wicking layer comprises bamboo or viscose fibres. In some embodiments the wicking layer comprises natural fibres such as cotton and bamboo which have been scoured and de-waxed to enhance their hydrophilicity.

In some embodiments the wicking layer comprises synthetic fibres comprising a hydrophilic surface. Base polymers for the synthetic fibres may include polyesters, polyamides, polyacrylates or polyolefins. In some embodiments the fibres comprise a hydrophilic surface which is applied using at least one of hydrophilic surfactants; hydrophilic plasma treatment; hydrophilic additives; hydrophilic binders and/or hydrophilic coatings. In some embodiments the wicking layer comprises man-made fibres that are inherently wettable such as fibres from regenerated cellulose including polynosics, modal, viscose, cuprammonium rayon, lyocell, veocel, and regenerated protein fibres such as silk. The fibres may comprise synthetic or regenerated fibres which comprisee non-circular fibre cross-sections to increase their absorbency, these may include: trilobal fibres, 4DG fibres (i.e. fibres comprising thermoplastic polymer, e.g. PET, PP or Nylon, which contain 6-10 grooves or channels along the longitudinal axis of the fibre), hexaflower fibres, ribbon fibres, triangular fibres and hollow fibres.

In some embodiments, the wicking layer comprises microfibres, which promote wicking by forming structures with small capillaries increasing the capillary forces driving the liquid flow. Microfibres are fibres with a diameter below 10 μm and are either extruded and drawn, formed by splitting of segmented pie bicomponent fibres of higher diameter into segments with microfibre diameter range, or ‘islands-in-the-sea’ bicomponent fibres where the matrix (sea) is dissolved and the microfibres (islands) form the structure.

In some embodiments, the wicking layer comprises a sheet of fibrous material having a fabric density of 20-60 g/m²; for example, 30-50 g/m². For example, a sheet of non-woven (e.g. spunlace) fibres having a fabric density of 20-60 g/m².

In an exemplary embodiment, the wicking layer comprises a sheet of parallel-laid spunlace cellulosic fibres, wherein the sheet has a fabric density of 20-60 g/m² and comprises 15-30 apertures per in² (i.e. ‘15-30 mesh’ or 2-5 apertures per cm²). In a further example, the wicking layer comprises a sheet of parallel-laid spunlace cellulosic fibres, wherein the sheet has a fabric density of 25-55 g/m² and comprises 20-25 apertures per in² (i.e. ‘20-25 mesh’ or 3-4 apertures per cm 2). For example, the wicking layer may comprise a sheet of parallel-laid spunlace bamboo or viscose fibres, wherein the sheet has a fabric density of 25-55 g/m² and comprises 20-25 apertures per in² (i.e. ‘20-25 mesh’ or 3-4 apertures per cm²).

Computer Implemented Method

The present invention provides a computer-implemented method of obtaining information related to an estimation of menstrual fluid quantity in a used sanitary napkin, the method comprising, the computer-implemented method comprising: obtaining or receiving first data comprising an image of a body-facing surface of a used sanitary napkin; the body-facing surface comprising indicia configured to facilitate the processing of data contained in an image of the body-facing surface; processing the first data to generate second data identifying a location of the indicia; processing the first data to generate a third data identifying an area of contrasting colour on the body-facing surface; and generating an output data related to an estimation of menstrual fluid quantity based at least on the second data and the third data.

The present invention also provides a non-transitory computer-readable storage medium comprising computer-executable instructions which, when executed by at least one processor, cause the at least one processor to: obtain or receive first data representing an image of a body-facing surface of a used sanitary napkin; the body-facing surface comprising indicia configured to facilitate the processing of data contained in an image of the body-facing surface; process the first data to generate second data identifying a location of indicia on the body-facing surface; process the first data to generate third data identifying a location of an area of contrasting colour on the body-facing surface; and generating an output data related to an estimation of menstrual fluid quantity based at least on the second data and the third data.

The present invention also provides a system for obtaining information related to an estimation of menstrual fluid quantity in a used sanitary napkin, the method comprising:
a) a sanitary napkin comprising a body-facing layer comprising a body-facing surface; a liquid-impermeable garment-facing layer; and an absorbent structure therebetween; and wherein the body-facing surface of the body facing layer comprises indicia configured to facilitate the processing of data contained in an image of the body-facing surface; and
b) a software application for performing a process comprising; obtaining or receiving first data representing an image of a body-facing surface of a used sanitary napkin; the body-facing surface comprising indicia configured to facilitate the processing of data contained in an image of the body-facing surface;
processing the first data to generate second data identifying a location of indicia on the body-facing surface;
processing the first data to generate third data identifying a location of an area of contrasting colour on the body-facing surface; and
generating an output data related to an estimation of menstrual fluid quantity based at least on the second data and the third data.

In some embodiments the second data comprises a distance between at least two indicium as represented in the image.

In some embodiments the third data comprises an area of the body-facing surface occupied by a material having a colour contrasting with the colour of the body-facing surface. In some such embodiments the colour contrasting with the colour of the body-facing surface is the colour of menstrual fluid (e.g. blood).

In some embodiments the estimating a quantity of menstrual fluid disposed on the sanitary-napkin comprises normalising the third data (i.e. the data identifying a location of contrasting colour on the body-facing surface) using the distance measured between at least two indicium; calculating a normalised area value for the region of contrasting colour; and converting the normalised area value to an output data, for example, by reference to a look-up table.

In some embodiments the generating an output data comprises at least one of;
a) reporting a quantity of menstrual fluid (for example, reporting a volume value of menstrual fluid);
b) reporting an arbitrary value representative of a quantity of menstrual fluid (for example, reporting a ‘number of teaspoons’ of menstrual fluid);
c) making a recommendation for one or more suitable feminine hygiene product(s); for example, recommending a product which is suitable for the quantity of menstrual fluid produced by the wearer.

In some embodiments the output data comprises at least one of; a quantity of menstrual fluid; an arbitrary value representing a quantity of menstrual fluid; and, a recommendation for one or more suitable feminine hygiene product(s). In some embodiments the arbitrary value representing a quantity of menstrual fluid is an integer or indication on a ‘scale of saturation’ range. For example, saturation of the sanitary napkin could be indicated using values from 1-5, with 1 being the lowest degree of saturation and 5 being the highest. In a further example, an indication of menstrual fluid quantity could be provided in terms of ‘teaspoons’ of menstrual fluid.

In some embodiments the first, third and/or output data further comprises a date and/or time value. In some embodiments, the computer implemented method further comprises storing at least one of the first, third and/or output data; for example, on a memory card/device (e.g. ROM and/or SD card) or in a cloud-based storage facility. When the first, third and/or output data further comprises a date and/or time value this may be used to provide information on the quantity of menstrual fluid produced at different stages of (or throughout the duration of) the wearer's menstruation. Alternatively, or in addition, this would facilitate recommendations of suitable feminine hygiene products for different stages (e.g. each day) of the wearer's menstruation, as the quantity of menstrual fluid varies throughout the duration of menstruation and different products may be appropriate at different stages.

In these embodiments the indicia may comprise any features of indicia described elsewhere herein.

### Exemplary Embodiments and Figures

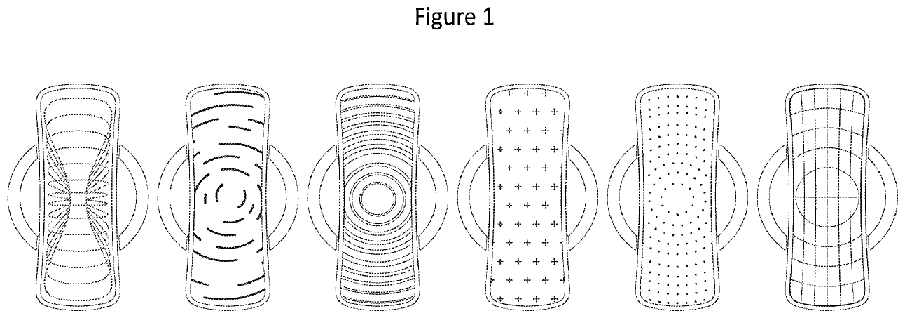

As shown in FIG. 1, the indicia on the body-facing surface of the sanitary napkin may form a wide variety of pre-defined patterns. A method of estimating a quantity of menstrual fluid absorbed into the sanitary napkin could comprise measuring the distance between indicia in order to normalise the value of a measured surface area of contrasting colour on the surface of the napkin.

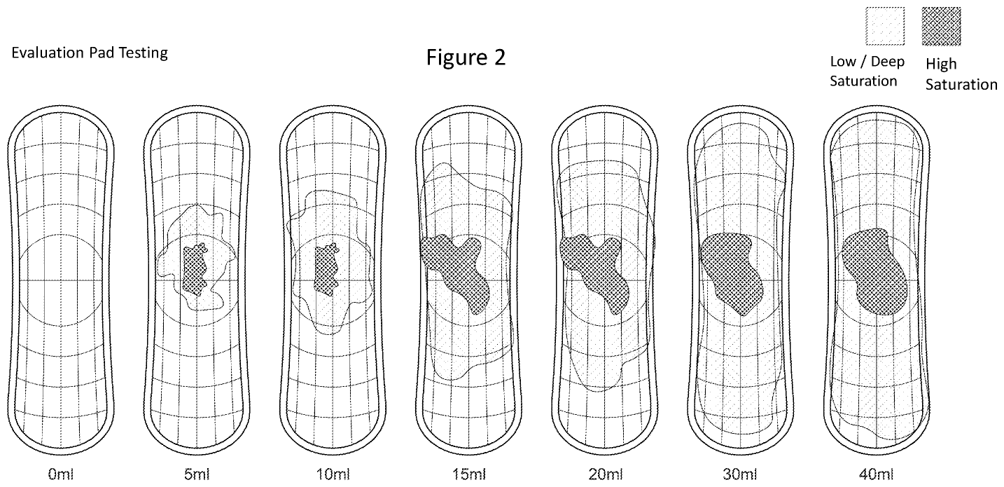

An example of this is shown in FIG. 2; in this case the indicia are presented in the form of lines. A method of estimating a quantity of menstrual fluid absorbed into the napkin could include measuring the distance between the lines and using the measured distance to normalise the measured value for a surface area of contrasting colour (representing the surface area of menstrual fluid) staining the body-facing surface of the napkin. The normalised surface area of menstrual fluid can then be converted to an estimate of menstrual fluid quantity (e.g. volume of menstrual fluid) using a look-up table.

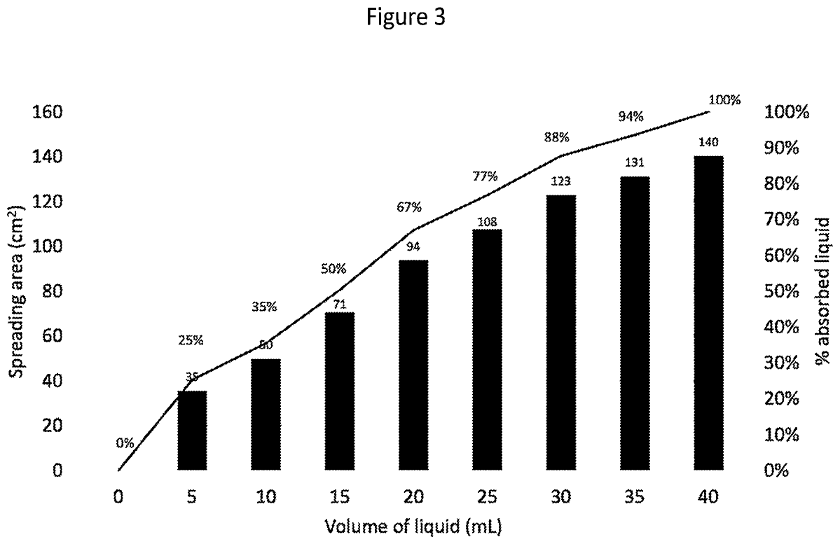

FIG. 3 shows a graph containing the results of a standard curve comparing spreading area (cm 3) with a set volume of liquid added to a standardized sanitary napkin (such as a sanitary napkin of the invention). This is an example of data which could be entered into a look-up table, by which a volume of menstrual fluid could be ascertained, based on the normalised surface area of menstrual fluid recognised in an image of the body-facing surface.

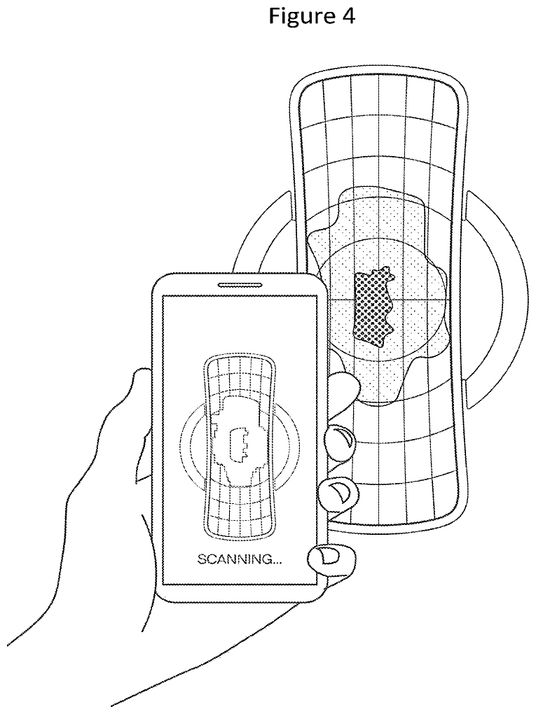

As is shown in FIG. 4, such an image could be taken and analysed using a smartphone app. The app could be configured to recognise the indicia on the body-facing surface of a sanitary napkin, as described elsewhere herein, and recognise an area of contrasting colour resulting from menstrual fluid staining the body-facing surface. The app would then use the indicia to normalise the surface area. Using the look-up table, the app could then report an estimate for a quantity of menstrual fluid contained in the napkin.

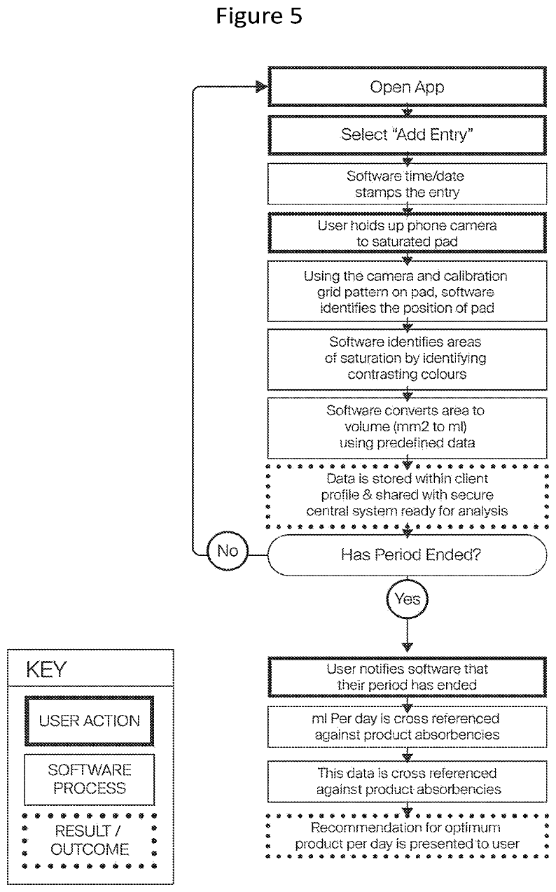

FIG. 5 contains a flow chart explaining how such a smartphone app could work: A user opens the app and selects a function indicating that they would like to make a data entry; the software time/date stamps the entry. The user holds up their phone camera to saturated sanitary napkin of the invention. Using the indicia provided on the body-facing surface of the sanitary napkin the software identifies a position of the sanitary napkin. The software also identifies areas of saturation by identifying colour contrast. The software converts area to volume mm² to mL using pre-defined data. The data is stored within client profile and shared with secure central system ready for analysis. The user notifies software that their period has ended. mL per day/input data is cross-referenced against product absorbencies and (optionally) a recommendation for optimum product per day is presented to the user.

### EXAMPLES

### Example 1—Exemplary Sanitary Napkin

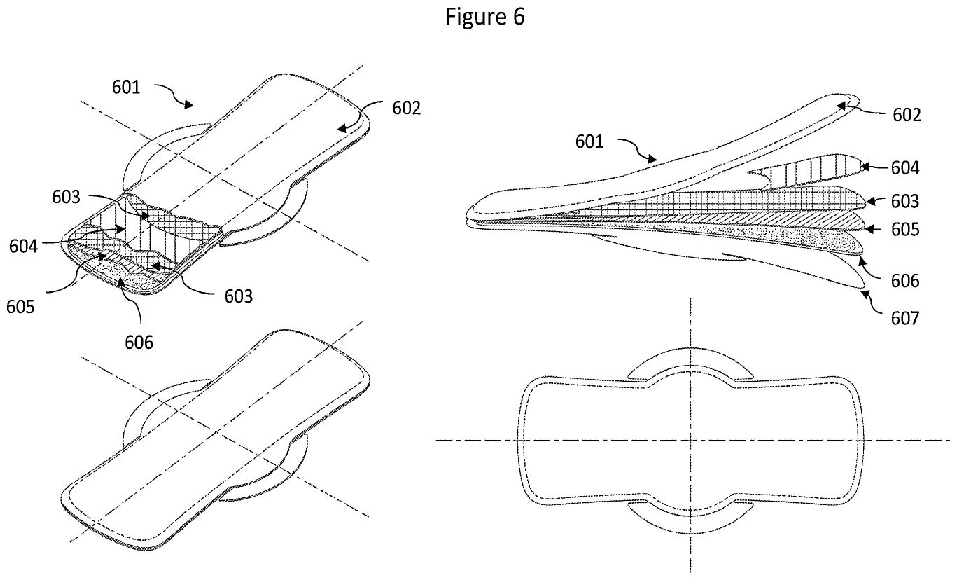

As shown in FIG. 6, an exemplary sanitary napkin 601 of the invention comprises a body-facing layer which comprises a top-sheet 602 and a barrier sheet 604. The napkin comprises a liquid impermeable garment-facing layer 607 and an absorbent structure 606 (also referred to as an absorbent core) between the body-facing layer and the garment-facing layer. Between the body-facing layer and the absorbent structure is a wicking layer 605. An ADL 603 may form part of the body-facing layer, or may be positioned between the barrier sheet 604 and the absorbent structure 606.

An exemplary sanitary napkin of the invention could comprise the components listed in Table 1 below:

TABLE 1

| Component | Material |
|---|---|
| Indicia | Styrene acrylate binder with white |
| | ink (hydrophobic evaluation lines) |
| | Superabsorbent coating with red |
| | ink (hydrophilic evaluation lines) |
| Top-sheet 602 | Hydrophilic PP SMS (8 g/m²) |
| Barrier sheet 604 | Polyethylene film (20 μm thickness) |
| ADL 603 | Carded air-through bonded PET/ |
| | biocomponent PET (35 g/m²) |
| Wicking layer 605 | Apertured viscose spunlace |
| Absorbent structure 606 | Pulp and SAP (250 g/m²) |
| Garment-facing layer 607 | PE film (20 μm thickness) |
| Adhesive | 3M spray adhesive |

### Example 2—Alternative Materials

It may be of interest to exchange materials in the sanitary napkins for compostable materials. Consequently, the inventors have investigated a variety of home and industrially compostable materials and exemplary materials for each component are provided in Table 2, below.

TABLE 2

| | | Recommended | |
|---|---|---|---|
| | Recommended | material | Further home |
| | material (home | (industrially | compostable |
| Component | compostable) | compostable) | materials |
| Top-sheet | Hydrophobic | Spunbond or | PHA |
| | cotton or | spunlace | spunbond/ |
| | Tencel | polylactic | spunlace |
| | spunlace | acid (PLA) | |
| ADL | Pulp + Binder | Air-through | Air laid cotton |
| | | bonded PLA | ADL |
| Garment-facing | Thermoplastic | Biodegradable | Bio-based/Home |
| layer | Starch (TPS) | polyethylene (PE) | compostable PE |

| Absorbent | Pulp/Potassium polyacrylate powder | |
|---|---|---|
| structure | | |
| Wicking layer | Apertured spunlace viscose/bamboo | |
| Adhesive | Rosin-based or | Bio-based |
| | Terpene based adhesives | polymer |
| | | Adhesives |

### Example 3—Liquid Wicking Rate (Capillarity) of Wicking Layer

A liquid wicking rate assessment of different wicking materials was carried out according to EDANA WSP 101.1.R0 (20) as is detailed above.

Each specimen was placed vertically in 15 mm of fluid (0.9% saline solution) and liquid rise (wicking distance) was recorded at 10, 30, 60 and 300 seconds.

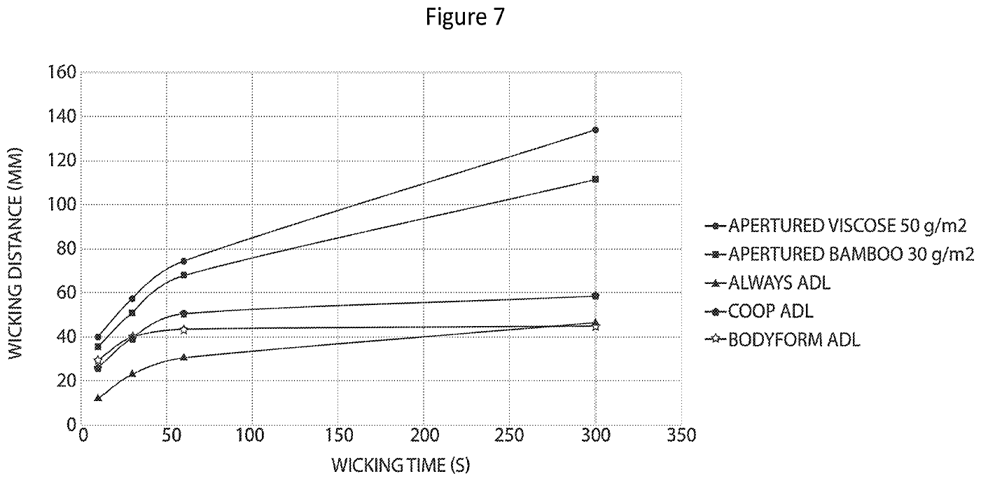

Test specimens as shown in the results of FIG. 7 were:
1. Spunlace apertured viscose 50 g/m²
2. Spunlace apertured bamboo 30 g/m²
3. Commercial ADL from an Always Ultra pad
4. Commercial ADL from a COOP branded pad
5. Commercial ADL from a Bodyform pad

Results of liquid wicking rate tests performed in accordance with the standard method are shown in FIG. 7. Apertured viscose and bamboo performed better or equivalently to the ADL layers of commercial sanitary napkins in terms of liquid wicking rate.

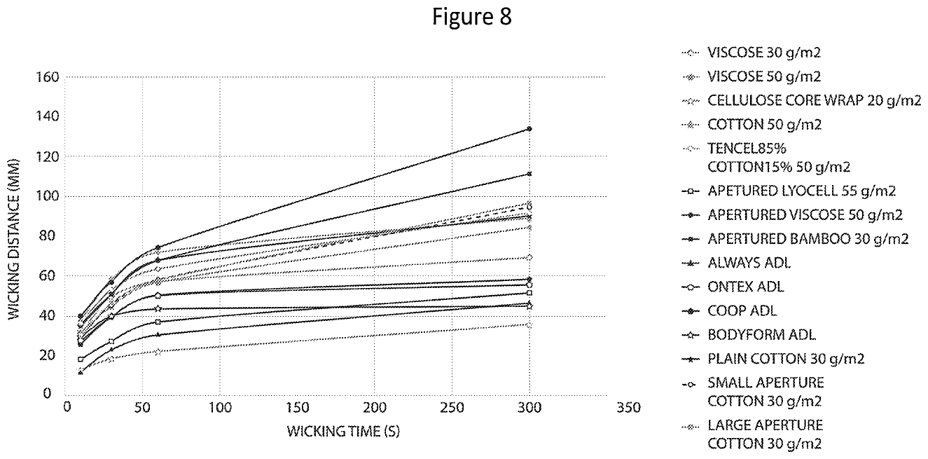

Further wicking materials were also tested for comparative purposes, using the same standard procedure. The results shown in FIG. 8 indicate that wicking layers prepared from a range of natural and man-made materials perform adequately in terms of wicking rate. The majority of wicking materials tested for use in the invention wicked fluid at a faster rate than the ADL layers of commercial sanitary napkins (Always ADL, Ontex ADL, COOP ADL and Bodyform ADL).

### Example 4—Liquid Absorptive Capacity of Wicking Layer

Experiments were undertaken to examine the liquid absorptive capacity of wicking layers comprising spunlace apertured viscose and spunlace apertured bamboo. The tests were carried out according to EDANA NWSP 101.1.RO0 (20)(as detailed above).

Samples were submerged below the surface of a 0.9% saline solution for 60 seconds, then hung freely vertical to drain for 120 seconds. The liquid absorptive capacity was then calculated from dry and final wet weights.

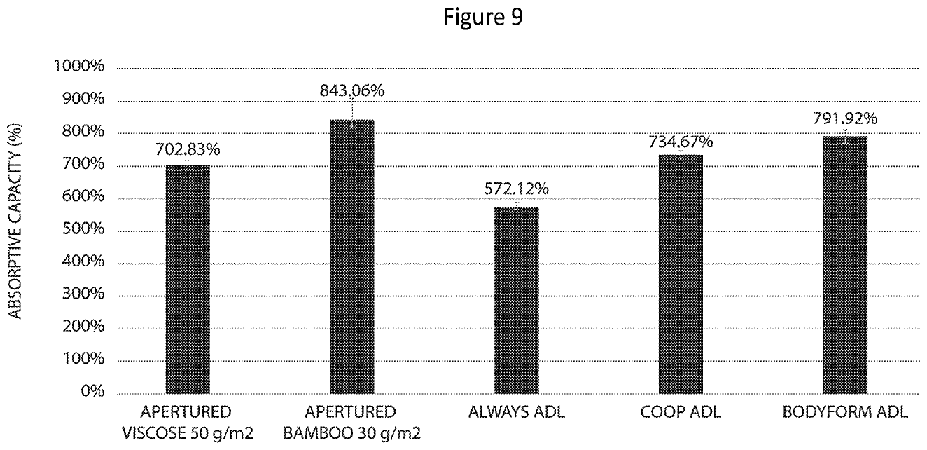

Test specimens as shown in results of FIG. 9 were:
1. Spunlace apertured viscose 50 g/m2
2. Spunlace apertured bamboo 30 g/m2
3. Commercial ADL from an Always Ultra pad
4. Commercial ADL from a COOP branded pad
5. Commercial ADL from a Bodyform pad

Results of liquid absorptive capacity tests performed in accordance with the standard method are shown in FIG. 9. Apertured viscose and bamboo performed better or equivalently to the ADL layers of commercial sanitary napkins in terms of liquid absorptive capacity.

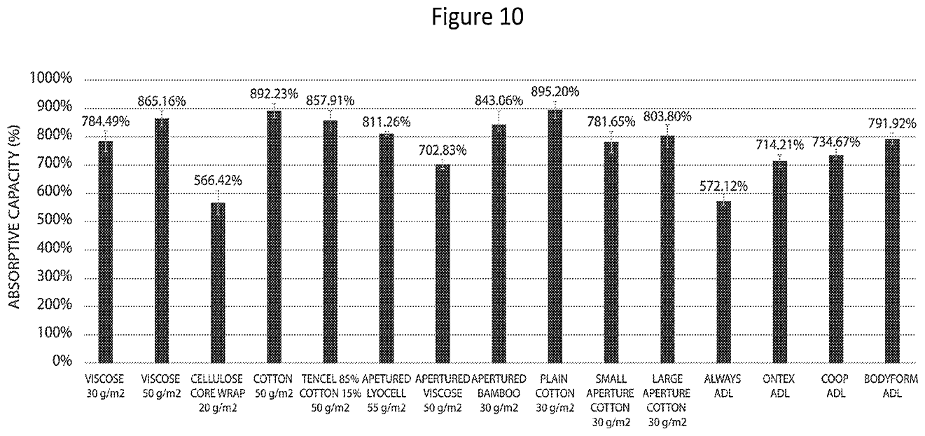

The absorptive capacity of further wicking materials was also tested for comparative purposes, using the same standard procedure. The results for these materials (shown in FIG. 10) indicate that wicking layers prepared from a range of natural and man-made materials perform adequately in terms of absorptive capacity. The majority of wicking materials tested for use in the invention had a higher liquid absorptive capacity than the ADL layers of commercial sanitary napkins (Always ADL, Ontex ADL, COOP ADL and Bodyform ADL).

### Example 5—Correlation Between Inlet Volume and Spreading Area

The skilled person will appreciate that there are numerous ways to estimate a quantity of menstrual fluid based on a measured surface area of a contrasting colour.

As an example, a look-up table may be prepared using a standard curve. To prepare a standard curve, a solution with similar consistency and flow properties to menstrual fluid is applied using a standardized procedure to a standardized sanitary napkin of the present invention to be absorbed by the sanitary napkin. The solution has a colour which contrasts with the colour of the body-facing surface of the sanitary napkin such that it can be visually detected on the body-facing surface. Once absorbed into the sanitary napkin, the solution stains the body-facing surface of the sanitary napkin leaving an area of contrasting colour to the colour of the body-facing surface. The surface area stained by the contrasting colour is identified and measured and this is equated with the quantity of solution applied to the sanitary napkin. The preceding steps are repeated for a range of quantities of the solution (e.g. 5-40 mL of liquid as shown in the graph of FIG. 3) and a look-up table for estimating quantity of solution based on the measured surface area of the contrasting colour is prepared.

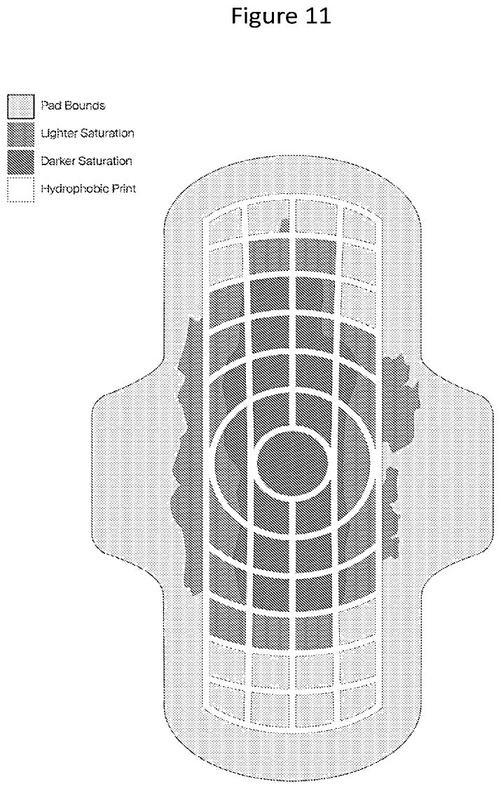

Referring to FIG. 11, there is shown a sanitary napkin having indicia on the body-facing surface with the liquid applied. In this exemplary embodiment, the indicia on the sanitary napkin are in the form of lines. The inventors found that reducing the frequency of lines or increasing the space between lines running horizontally across the napkin is advantageous such that the lines do not prevent the longitudinal spread of the fluid.

The lines are of hydrophobic material, which repels the menstrual blood and therefore the blood does not stain the hydrophobic lines, as shown in FIG. 11. A suitable material could be OC-Bioorganic (from supplier OrganoClick), which is a hydrophobic biobased binder comprising modified biopolymers and natural plant compounds. The hydrophobic nature of the indicia creates a very defined, striking overall visual effect and displays in high contrast when the sanitary napkin is saturated with blood. This is advantageous in that it helps the users to manually align or for the application to self-correct the capture image. The inventors have, through their own research and testing, found that the hydrophobic lines provide good clarity and accuracy when translating saturation of menstrual blood on the napkin through to the smartphone application.

Furthermore, the cells and/or segments formed on the body-facing surface of the sanitary napkin by the hydrophobic lines have a channelling effect when blood is applied. Each cell and/or segment pools the liquid, such that liquid is prevented from moving across the top-sheet by the hydrophobic indicia and is channelled directly into the absorbent core and along the length of the sanitary napkin at the point of entry. As shown in FIG. 11, the point of entry, which is usually the centre of the napkin, has a darker shade and which indicates a deeper saturation of blood. The cells towards the peripheral of the sanitary napkin have a lighter shade which indicates a lighter saturation of blood. This arrangement helps to control the spread and encourages the liquid to be drawn into the core of the napkin before spreading across the napkin, thereby preventing unwanted flow of fluid across the top-sheet of the sanitary napkin, causing leakage. The inventors found that the hydrophobic lines also prevent horizontal failure by preventing the liquid from spreading and soaking the peripheral of the sanitary napkin, such as the wings. This contrasts with where the napkin does not contain hydrophobic indicia, the liquid spreads horizontally across the sanitary napkin much quicker and much further.

The hydrophobic lines are preferably a light colour to contrast with the blood, and preferably contain a white dye/ink/pigment to contrast with the red colour of menstrual blood. Optionally, the body-facing surface of the napkin further comprises a further indicia on or around the outer boundary of the hydrophobic lines, preferably of a dark or coloured ink to help refine and optimise the indicia pattern further, and to facilitate the aligning of the sanitary napkin especially when there is low or no saturation of blood. This is because most of the sanitary napkins in the market are of white colour, and white lines on a white napkin could be difficult for the camera of a smartphone to identify. In this aspect, the sanitary napkin would have indicia comprising hydrophobic material to help evaluate the amount of menstrual blood, and further indicia to help identify the boundary of the napkin itself. The further indicia are preferably visible against the surface of the napkin, and so can be darker or coloured to contrast with the surface of the napkin. The further indicia are useful to help identify the boundary of the napkin, such as around the periphery of the absorbent area of the napkin.

Preferably, a smartphone application is used together with the sanitary napkin to provide an improved diagnostics and effective fertility-preserving medical treatments for users who experience problematic menstrual bleeding. The smartphone application analyses the volume of fluid lost, the duration or the number of days of bleeding in each cycle, the length of each cycle, and the regularity or variation in time between each cycle. The application can further collect data from scans to detect and analyse clots size and frequency and/or leakage as well as provide period and pill tracking/notification function. More preferably, the application has a manual editor that allows users to both ‘un-identify’ areas that are mis-identified, as well as to identify areas that were missed. The application further has a manual review, allowing users to confirm the presence of clots on the surface of the sanitary napkin.

In a specific example, the application has some on-boarding questions for the users including some general information about the user's age, gender, ethnicity, height, weight, childbirth, and the reason for evaluation, as well as some medical information such as medication, medical conditions, birth control, change of menstrual cycle due to vaccination or infectious diseases such as COVID, and their period experience so far. Based on the information provided by the users and the scan data collected from each menstrual cycle, the application can provide a useful, informative analysis of their menstruation to help users make better, well-informed choices and to help them improve their experience. The application can further recommend optimum products based on absorbency on each day of the cycle by analysing the volume of fluid lost in past cycles, and provide symptoms checkers, as well as early detection and diagnosis of menstrual disorders, for example by collecting the blood sample on the sanitary napkin for laboratory analysis.

The above embodiments are to be understood as illustrative examples of the invention. Further embodiments of the invention are envisaged. It is to be understood that any feature described in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

What is claimed is:

1. A sanitary napkin comprising a body-facing layer comprising a body-facing surface;
a liquid-impermeable garment-facing layer; and
an absorbent structure therebetween;
wherein the body-facing surface comprises indicia configured to facilitate the processing of data contained in an image of the body-facing surface and wherein the indicia comprise hydrophobic material.

2. The sanitary napkin of claim 1, wherein the indicia are visually detectable when menstrual fluid comprising blood, or blood is present in the sanitary napkin.

3. The sanitary napkin of claim 1, wherein the indicia are configured to interact with menstrual fluid comprising blood, or blood, so as to become and/or remain visually detectable when menstrual fluid is present in the sanitary napkin.

4. The sanitary napkin of claim 1, wherein the indicia comprise lines or spots.

5. The sanitary napkin of claim 1, wherein a portion of the indicia comprise hydrophilic material and a portion of the indicia comprise hydrophobic material.

6. The sanitary napkin of claim 4, wherein the indicia are configured to be recognized by a software application, to calibrate the position and/or size of the body-facing surface in an image of the sanitary napkin.

7. The sanitary napkin of claim 1, wherein the indicia are configured to facilitate an estimation of a quantity of menstrual fluid contained in the sanitary napkin after use.

8. The sanitary napkin of claim 1, wherein the body-facing surface comprises further indicia configured to help identify the boundary of the sanitary napkin, wherein the further indicia comprise a dark or coloured ink.

9. A computer-implemented method for obtaining information related to an estimation of menstrual fluid quantity in a used sanitary napkin, the method comprising:
obtaining or receiving first data representing an image of a body-facing surface of the used sanitary napkin; the body-facing surface comprising indicia configured to facilitate the processing of data contained in an image of the body-facing surface, and wherein the indicia comprise hydrophobic material;
processing the first data to generate second data identifying a location of indicia on the body-facing surface;
processing the first data to generate third data identifying a location of an area of contrasting colour on the body-facing surface; and
generating an output data related to an estimation of the menstrual fluid quantity based at least on the second data and the third data.

10. The method of claim 9, wherein the second data comprises a measured distance between at least two indicia as represented in the image.

11. The method of claim 9, wherein the third data comprises an area of the body-facing surface occupied by a material having a colour contrasting with the colour of the body-facing surface.

12. The method of claim 9, wherein the third data is normalised relative to the second data.

13. The method of claim 9, wherein the step of generating an output data comprises at least one of;
a) reporting a value representative of a quantity of menstrual fluid;
b) making a recommendation of one or more suitable feminine hygiene product(s); and
c) an arbitrary designation representing quantity.

14. The method of claim 9, wherein the first, third and/or output data further comprises a date and/or time value.

15. A non-transitory computer-readable storage medium comprising computer-executable instructions which, when executed by at least one processor, cause the at least one processor to:
obtain or receive first data representing an image of a body-facing surface of a used sanitary napkin; the used sanitary napkin comprising a body-facing surface; the body-facing surface comprising indicia configured to facilitate the processing of data contained in an image of the body-facing surface, wherein the indicia comprise hydrophobic material;
process the first data to generate second data identifying a location of indicia on the body-facing surface;
process the first data to generate third data identifying a location of an area of contrasting colour on the body-facing surface; and
generate an output data related to an estimation of menstrual fluid quantity based at least on the second data and the third data.
